(19) United States
(12) Patent Application Publication
(10) Pub. No.: US 2022/0218565 A1
(43) Pub. Date: Jul. 14, 2022

(54) ADAPTIVE PRIMARY PACKAGING FOR THERAPEUTIC SOLUTIONS

(71) Applicant: WEST PHARMACEUTICAL SERVICES, INC., Exton, PA (US)

(72) Inventor: Lawton Edward LAURENCE, Chester Springs, PA (US)

(21) Appl. No.: 17/611,948

(22) PCT Filed: May 14, 2020
(86) PCT No.: PCT/US2020/032881

§ 371 Date: Nov. 17, 2021

Related U.S. Application Data
(60) Provisional application No. 62/849,215, filed on May 17, 2019.

(51) Int. Cl. A61J 1/06

(52) U.S. Cl. CPC A61J 1/062; A61J 1/067

(57) ABSTRACT

Various container assemblies for containing a therapeutic liquid are provided. A first container assembly comprises a frame having a proximal end and a distal end and includes a plurality of axially extending legs, a collar attached to the plurality of axially extending legs at the proximal end of the frame, and a collapsible film covering the frame and attached to the distal end of the frame and at least a portion of the collar. A second container assembly for a therapeutic liquid comprises a collar and a collapsible film. The collar may comprise a plurality of hingedly connected segments configured to switch from an open configuration to a closed configuration. The collapsible film may be configured as a pouch having an open end attached to one end of the collar.

### CROSS-REFERENCE TO RELATED APPLICATION

This application claims the benefit of and priority to U.S. Provisional Application Ser. No. 62/849,215, filed on May 17, 2019; the content of which is incorporated herein by reference in its entirety.

### BACKGROUND

Various embodiments disclosed herein are directed to container assemblies. More specifically, the various embodiments are directed to container assemblies that are flexible and easily adaptable to various uses as primary packaging for therapeutic solutions.

The design of the primary container for holding therapeutic medicines in liquid form encounters several challenges. For example, the volume of medicine delivered and tested to clinical patients during early stage development may need be either increased or decreased during the development and/or approval process during the later stages of development. As a result, the form of the primary container used to hold and/or deliver the medicine may need to be re-designed. This may not only affect the design of the delivery device that carries the primary container, but also the filling equipment used to fill the primary container.

Thus, there is a need for improved primary packaging for therapeutic solutions that may be easily adaptable to various environments during the drug development process, as well as manufacturing processes when the drug is ultimately approved for use.

### SUMMARY

In one aspect, a container assembly for a therapeutic liquid comprises a frame, a collar, and a collapsible film. The frame may have a proximal end and a distal end and comprise a plurality of axially extending legs. The collar may be attached to the plurality of axially extending legs at the proximal end of the frame. The collapsible film may cover the frame and attach to the distal end of the frame and at least a portion of the collar.

In another aspect, a container assembly for a therapeutic liquid comprises a collar and a collapsible film. The collar may comprise a plurality of hingedly connected segments configured to switch from an open configuration to a closed configuration. The collapsible film may be configured as a pouch having an open end attached to one end of the collar.

These and other aspects of the various embodiments disclosed herein will be apparent in view of the following description.

### BRIEF DESCRIPTION OF THE SEVERAL VIEWS OF THE DRAWINGS

Various aspects and embodiments of the present concepts disclosed herein will be described with reference to the following figures. It should be appreciated that the figures are not necessarily drawn to scale. The figures depict one or more implementations in accord with the present concepts, by way of example only, not by way of limitations. In the figures, like reference numerals refer to the same or similar elements.

FIG. 1 is a top perspective exploded view of a container assembly according to a first embodiment.

FIG. 2A is a top perspective view of the first embodiment in an assembled condition.

FIG. 2B is a front view of the first embodiment in the assembled condition.

FIG. 2C is a side view of the first embodiment in the assembled condition.

FIG. 2D is a side view of the first embodiment, wherein the film-formed container is in a collapsed condition.

FIG. 2E is top perspective view of the first embodiment in combination with a needle guard.

FIG. 2F is a partial top perspective view of proximal end of a stopper collar of the first embodiment.

FIG. 3A is a side view of a container assembly according to a second embodiment.

FIG. 3B is a front view of the second embodiment.

FIG. 4 is a top perspective view of a container assembly according to a third embodiment.

FIG. 5A is a partial cross-sectional side view of a proximal end of a container assembly according to a fourth embodiment.

FIG. 5B is a partial cross-sectional side view of a proximal end of a container assembly according to a fifth embodiment.

FIG. 5C is a top perspective view of a closure that may be included in a container assembly.

FIG. 5D is a partial top perspective view of a proximal end of a container assembly with the closure of FIG. 5C.

FIG. 6A is a top perspective view of a container assembly according to a sixth embodiment having a collar in an open configuration.

FIG. 6B is a top perspective view of the sixth embodiment in a closed configuration.

FIG. 6C is a top perspective view of a seal portion of the sixth embodiment.

FIG. 6D is a partial top perspective view of the proximal end of the sixth embodiment illustrating the position of the seal portion within a collar.

FIG. 6E is a partial top perspective view of the proximal end of the sixth embodiment, wherein the collar is in the open configuration.

FIG. 6F is a partial top perspective view of the proximal end of the sixth embodiment, wherein the collar is in the closed configuration.

FIG. 6G is a magnified partial view of FIG. 6F.

FIG. 7A is a top perspective front view of a seventh embodiment, wherein the collar is in the open configuration.

FIG. 7B is a top perspective front view of the seventh embodiment, wherein the collar is absent.

FIG. 7C is a top perspective front view of a seal portion of the seventh embodiment.

FIG. 7D is a top perspective front view of the collar of the seventh embodiment.

FIG. 7E is a top perspective rear view of the collar of the seventh embodiment.

FIG. 7F is a top perspective side view of the collar of the seventh embodiment.

FIG. 8A is a top perspective view of a container assembly according to an eighth embodiment.

FIG. 8B is a top perspective view of a closure included in the container assembly of the eighth embodiment.

FIG. 8C is a top perspective view of the eighth embodiment excluding the closure.

FIG. 9A is a top perspective view of a container assembly according to a ninth embodiment.

FIG. 9B is a side view of the ninth embodiment.

FIG. 10A is a front plan view of a container assembly according to a tenth embodiment.

FIG. 10B is a front plan view of the collar and frame of the tenth embodiment.

FIG. 10C is a side view of FIG. 10B.

FIG. 10D is a bottom perspective view of the FIG. 10B.

### DETAILED DESCRIPTION

Certain terminology is used in the following description for convenience only and is not limiting. The words “lower,” “bottom,” “upper” and “top” designate directions in the drawings to which reference is made. The words “inwardly,” “outwardly,” “upwardly” and “downwardly” refer to directions toward and away from, respectively, the geometric center of the liquid transfer device, and designated parts thereof, in accordance with the present disclosure. Unless specifically set forth herein, the terms “a,” “an” and “the” are not limited to one element, but instead should be read as meaning “at least one.” The terminology includes the words noted above, derivatives thereof and words of similar import.

It should also be understood that the terms “about,” “approximately,” “generally,” “substantially” and like terms, used herein when referring to a dimension or characteristic of a component of the disclosure, indicate that the described dimension/characteristic is not a strict boundary or parameter and does not exclude minor variations therefrom that are functionally similar. At a minimum, such references that include a numerical parameter would include variations that, using mathematical and industrial principles accepted in the art (e.g., rounding, measurement or other systematic errors, manufacturing tolerances, etc.), would not vary the least significant digit.

It will also be appreciated by those skilled in the art that modifications may be made to the exemplary embodiments described herein without departing from the invention. Structural features of systems and apparatuses described herein may be replaced with functionally equivalent parts. Moreover, it will be appreciated that features from the embodiments may be combined with each other without departing from the disclosure.

Designs are disclosed for an adaptive primary container for therapeutic solutions suitable for high-volume manufacturing. The container may incorporate means for using traditional elastomeric closure materials and flexible-tube construction. A substantially rigid stopper collar serves a purpose similar to a flange of a syringe, allowing the container to be disposed within a traditional tub-and-nest format for introduction into fill-finish equipment. An element, such as a structural protrusion or frame, may extend from the stopper collar to the di stat end of the container. The protrusion may impart structural stability to the container that would not otherwise exist, A film may be sealed and attached to the stopper collar to establish a cavity in which the therapeutic solution is stored. An elastomer placed within the stopper collar provides for container closure integrity and in certain embodiments may be applied with traditional stoppering equipment.

Embodiments of the presently disclosed devices may achieve one or more of the following main aspects:
1. Introducing a primary container for pharmaceutical agents that is appropriate for long-term drug contact and permits a greater range of fill volumes while minimizing residual volume not delivered to the patient,
2. Allowing for delivery devices that accept a wider range of deliverable volumes while minimizing the size of the delivery device;
3. Permitting the same primary container to be utilized for early stage drug development and seamlessly incorporated into existing installed fill-finish equipment; and
4. Providing for a means of establishing structural integrity of the container without substantially reducing the advantages of the flexible cavity.

In certain embodiments, a container assembly according to the present disclosure may lend itself to current syringe-style filling practices. For example, a container assembly according to the present disclosure may not require significant changes to an existing installed base of automated equipment on a liquid filling line. In certain embodiments, a container assembly according to the present disclosure would support or would allow filling according to current high-volume practices.

In certain embodiments, a container assembly according to the present disclosure is adapted for incorporation and acceptability to current high-volume fill-finish equipment. Embodiments of the devices disclosed herein are compatible with syringe-style filling while maintaining the benefits of flexible-walled packaging.

In certain embodiments, a container assembly according to the present disclosure permits traditional, syringe-style fill-finish. For example, a container assembly according to the present disclosure may receive a stopper in similar fashion to a prefilled syringe. The container assembly may have structural stability provided by way of a substantially rigid frame. In certain embodiments, a container assembly according to the present disclosure can be rotated at high revolutions per minute consistent with standard procedures for particle inspection, in some embodiments, the container assembly may also be provided with a means to establish container closure integrity by a collapsing mechanism or portion, thus reducing or minimizing the size of the field container. In certain embodiments, a container according to the present disclosure incorporates an integrated needle for delivery in a manner similar to a prefilled syringe. Incorporating an integrated needle may reduce the number of steps that must be performed by the user in using the device.

In certain embodiments, a container assembly according to the present disclosure provides an increased range of fill volumes considering the size of the container, compared to alternative containers. This may enable flexibility during development of the medication as the dose size is determined through clinical trials. In certain embodiments, a container assembly according to the present disclosure provides greater flexibility, permitting incorporation into a variety of injection modalities without changing the primary packaging. For example, the container assemblies according to the present disclosure may have a smaller size for a given volume of injection than alternative containers. Container assemblies according to the present disclosure may also permit on-body-type injections without requiring transferring contents between containers at or prior to the time of use.

The container assemblies according to the present disclosure may be used for the storage of therapeutic solutions from tilling to patient administration. The process of filling may be substantially similar to that of a prefilled syringe. The containers may be supplied equipment in a tub-and-nest format to a site with appropriate fill-finish equipment. Once extracted from the secondary packaging, the containers may be filled by peristaltic or rotary motion pumps. Following filling of the container, container closure may be established using an elastomeric closure, such as a stopper, or using a collapsible collar described in greater detail below.

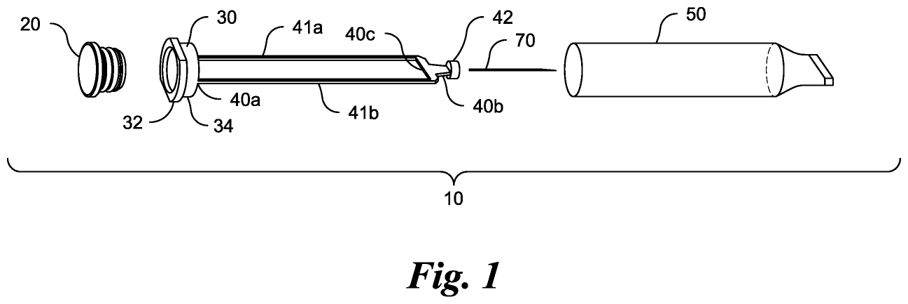

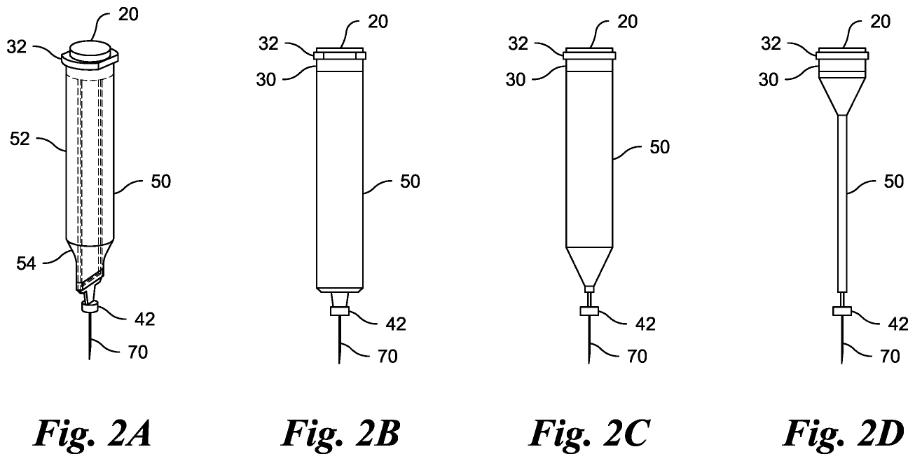

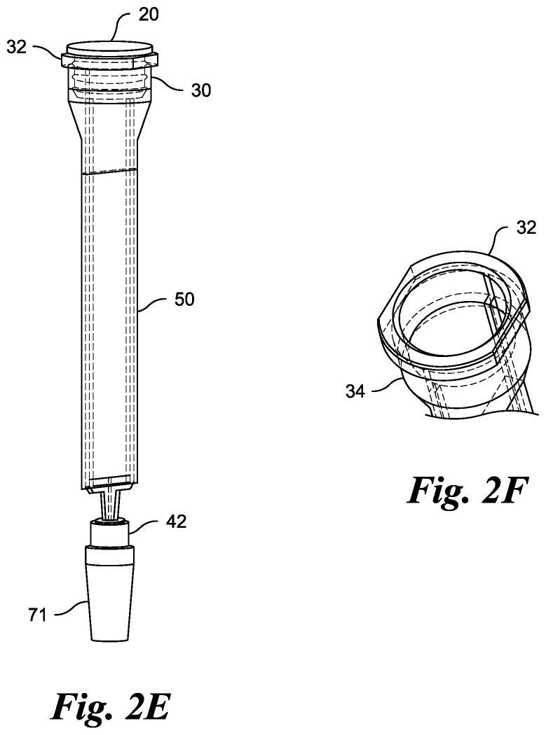

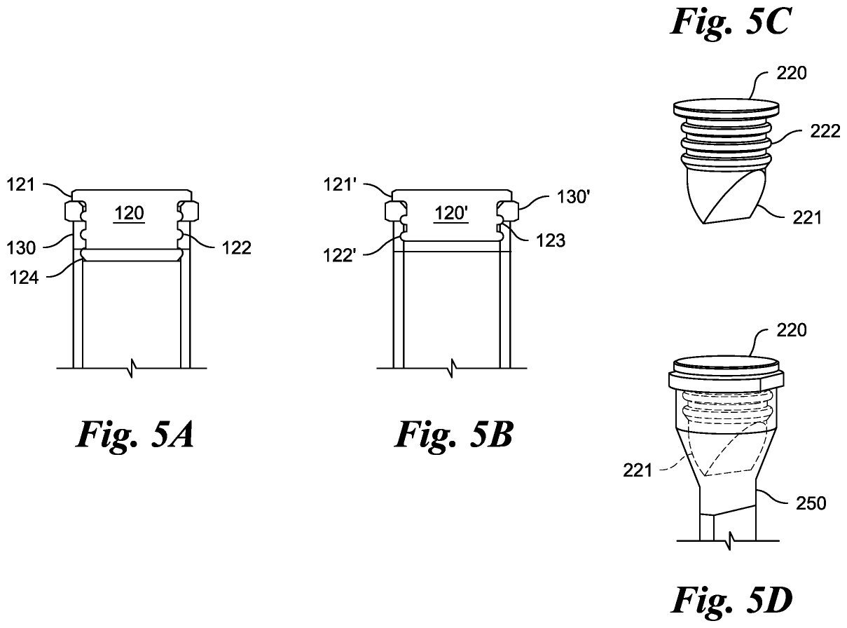

Referring now to FIGS. 1 to 2F, an example of a container assembly 10 according to a first embodiment is illustrated. In FIG. 1, which is an exploded view, the container assembly 10 includes a stopper 20. The stopper 20 is preferably elastomeric and may include one or more securing features, for example, the radial ridges and captivating ridge discussed below with respect to FIGS. 5A and 5B, to prevent displacement of the stopper 20 after insertion into an opening within a stopper collar 30 of the container assembly, as illustrated in the assembled view in FIG. 2A. The stopper collar 30 preferably is attached to a generally rigid frame 40. In the embodiment of FIG. 1, the frame 40 is generally rectangular having two parallel legs 41a, 41b. The proximal end 40a of the legs 41a, 41b are attached to the stopper collar 30 while the distal end 40b of the legs 41a, 41b are joined to an end portion 40c of the frame 40. The end portion 40c is preferably perpendicular to the parallel legs 41a, 41b. In some embodiments the stopper collar and frame may be formed as a unitary piece. In other embodiments, the stopper collar and frame may be separate pieces that are joined either mechanically with a snap fit, for example, or fused together.

The container assembly 10 further comprises a film 50 that is preferably attached to at least a portion of the stopper collar 30 and/or the frame 410, preferably the end portion 43 of the frame. The film 50 is preferably transparent to allow manual or automated inspection of the contents of the container assembly 10 after filling. Upon inserting the stopper 20 into the stopper collar 30, the container assembly 10 is preferably sealed to prevent the escape of any liquids contained therein.

The stopper collar 30, the frame 40, and the stopper 20 may be formed of polymer materials known in the art. For example, the stopper collar 30 and frame 40 may be made from cyclic olefin polymer, cyclic olefin copolymer, polypropylene, glass, or other suitable material, if of a rigid type, as well as combinations thereof. The stopper 20 is preferably elastomeric and may be made from one or more of butyl rubbers, thermoplastic elastomers, thermoplastic urethanes, or other suitable materials.

The stopper collar 30 optionally includes a flange 32 located at a proximal end of the main portion 34 of the stopper collar 30 and extends radially to a distance greater than the outer diameter of the main portion 34. The flange 32 may help to adapt the container 10 for use in tub-and-nest arrangements used in fill operations. The distal end portion 40c of the generally rectangular frame 40 optionally includes a needle huh 42 for accommodating a needle 70. The container assembly 10 may also optionally include a needle guard 71, as illustrated in FIG. 2E, for preventing needle sticks. The needle 70 and needle hub 42 preferably extend coaxially with the longitudinal axis of the container assembly 10. As previously noted, the film 50 is attached, preferably fused, to the main portion 34 of the stopper collar 30 and may be fused to a distal portion of the generally, rectangular frame 40, such as the distal end portion 40c thereof.

When the rectangular frame 40, the needle 70, and the film 50 are assembled, the assembled container 10, as seen in FIG. 2A, forms a closable cavity for holding a medicine or therapeutic solution. When the container assembly 10 is in the assembled condition and filled with a therapeutic solution, the film 50 may comprise a generally cylindrical proximal portion 52 and a tapered distal portion 34. The tapered distal portion 54 may help to reduce the amount of residual therapeutic solution or mixture that remains in the container assembly 10 when the contents are emptied or after injection into a patient due to imperfect emptying of the container 10. After filling the container, the stopper 20 is secured within the stopped collar 30 to form a sealed container that prevents leaking of any therapeutic liquid, and preferably prevents the ingress of any undesired contaminants into the container.

As previously noted, the container assembly 10 of FIG. 2A is in a filled condition, wherein the container has a therapeutic liquid stored therein. In the filled condition, the film 50 may form a generally cylindrical or other expanded form having a first volume. In order to expel the liquid within the container assembly, the flexible outer film may be manually or mechanically compressed causing the therapeutic liquid to exit the needle through the needle hub. When the container 10 is empty after use, the film 50 may flatten and conform to the shape of the internal frame, e.g. forming a rectangular shape of relatively small height or thickness, and having a second, lesser volume, (FIG. 2D).

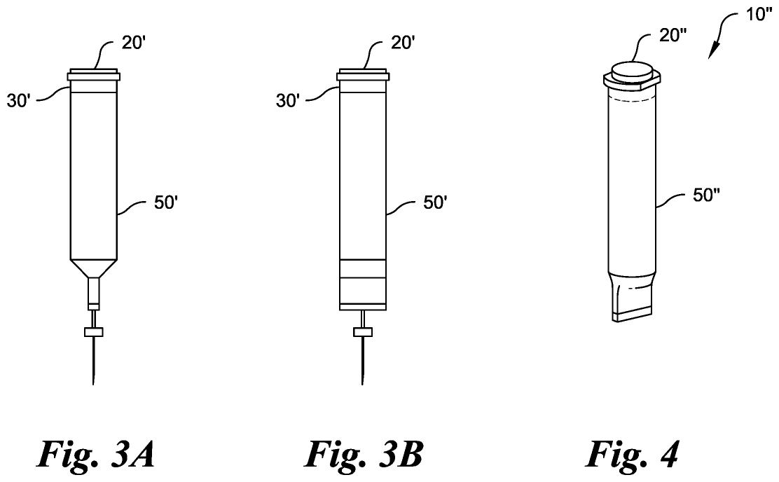

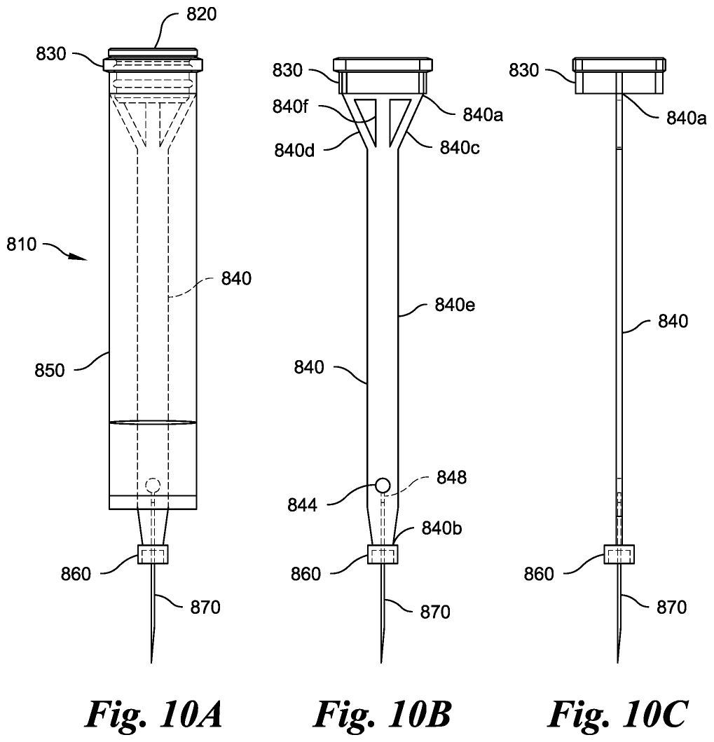

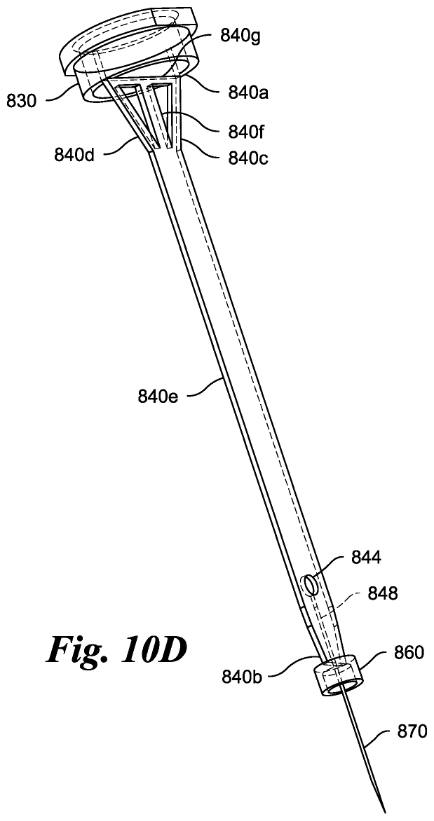

Referring to FIGS. 3A and 3B, another embodiment of the container assembly is illustrated. The container assembly may include a collar 30′ in which a stopper 20′ is inserted, as well as a flexible film 50′ in the shape of a container having a proximal end attached to a portion of the collar 30′ and a distal end that is welded or fused closed. The length of the weld may be selected to ensure closure integrity and prevent any leakages. The proximal end of the film 50′ may also be welded to a portion of an internal frame. For example, the container assembly may further comprise a V-shaped frame, such as the frame discussed in greater detail below with respect to FIGS. 10A-10D.

As previously noted, the inclusion of a needle hub and needle in the container assembly is optional. In some embodiments, such as the container assembly 10″ of FIG. 4, the assembly may include a stopper 20″, stopper collar 30″, and flexible film 50″ attached to an internal frame (not shown), such as those previously described; but, the needle hub and needle may be excluded. In order to extract the liquid stored within the container assembly 10″ of FIG. 4, a syringe or pump having a sufficiently long needle capable of penetrating through the thickness of the stopper 20″ may be inserted to access the internal contents of the container, for example.

In order to prevent displacement of the stopper and maintain a sealed container, the stopper may include one or more securing features. For example, referring to FIG. 5A, the proximal end of the stopper 120 may include a top flange portion 121. The diameter of the top flange portion 121 is preferably greater that than the inner diameter of the opening in the stopper collar, such that the bottom surface of the top flange portion 121 will abut the top surface of the stopper collar 130 when the stopper 120 is fully inserted into the opening of the stopper collar 130. The stopper 120 may also be provided with a wedge-like flange 124 at the distal end of the stopper 120. The wedge-like flange 124 may have an inclined leading edge that allows for insertion of the stopper 120 into the opening of the stopper collar 130, as well as a top edge that is parallel with the bottom surface of the stopper collar 130. The height of the stopper 120 may be selected, such that the top edge of the wedge-like flange 124 abuts against the bottom surface of the collar 130 when the top flange portion 121 contacts the top surface of the collar 130, thereby captivating the stopper 120 within the collar 130. The stopper 120 may further include one or more annular ridges 122 about the main portion of the stopper 120 between the top flange 121 and bottom wedge-like flange 124. The ridges 122 may have a diameter that is slightly greater than the opening of the collar 130 in order to form a seal between the ridges 122 and inner circumferential surface of the collar 130.

In another embodiment, a stopper 120′ as illustrated in FIG. 5B may again include a top stopper flange 121′ and one or more annular ridges 122′; however, instead of relying on a bottom wedge-like flange to captivate the stopper, the stopper collar 130 may be provided with annular grooves 123 in the inner circumferential surface of the collar 130′. The size, shape, and location of the grooves 123 may be selected to mate with the annular ridges 12′ of the stopper 120′ when the stopper 120′ is fully inserted and the top flange 121′ contacts the top surface of the collar 130′; thereby, captivating the stopper 120′ within the collar 130′.

FIG. 5C depicts a stopper 220 for minimizing dead-space volume within the container, i.e. minimizing the volume within the container wherein therapeutic liquid will potentially remain within the container and may not be capable of being expelled. The stopper 220 comprises a tapered distal end 22 and may further include one or more optional radial ridges 222 for sealing against the inner circumferential surface of corresponding stopper collar; as previously described. Referring to FIG. 5D, when the outer flexible film 250 is collapsed in order to expel the liquid contents of the container, the flexible film 250 is capable of conforming closely to the shape of the tapered distal end 221 of the stopper 220, thereby reducing the potential of any dead-space volume. The result is a reduction in the potential volume of residual therapeutic liquid remaining in the container after use.

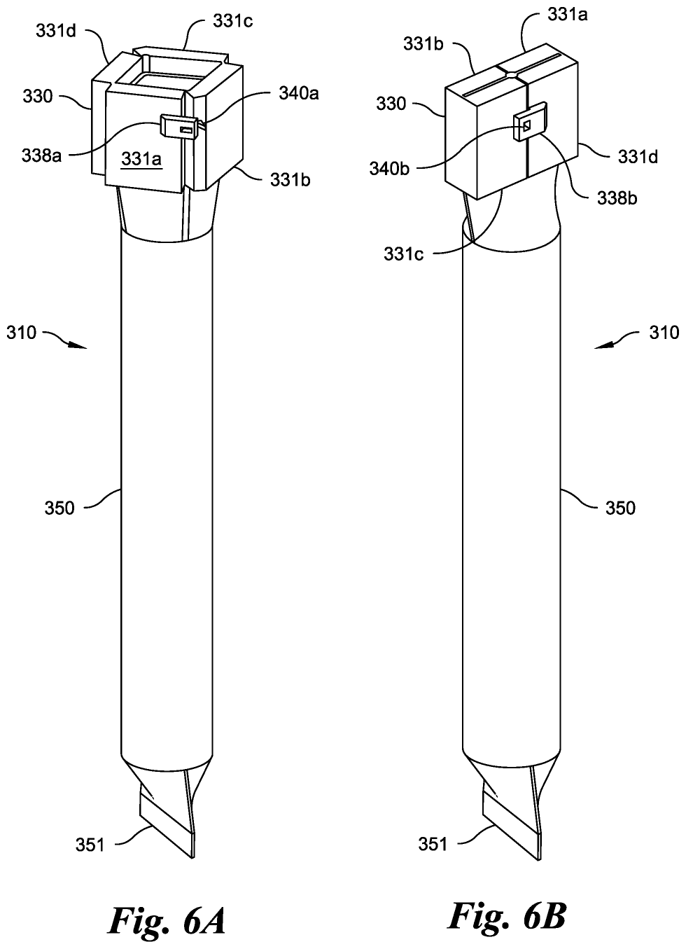

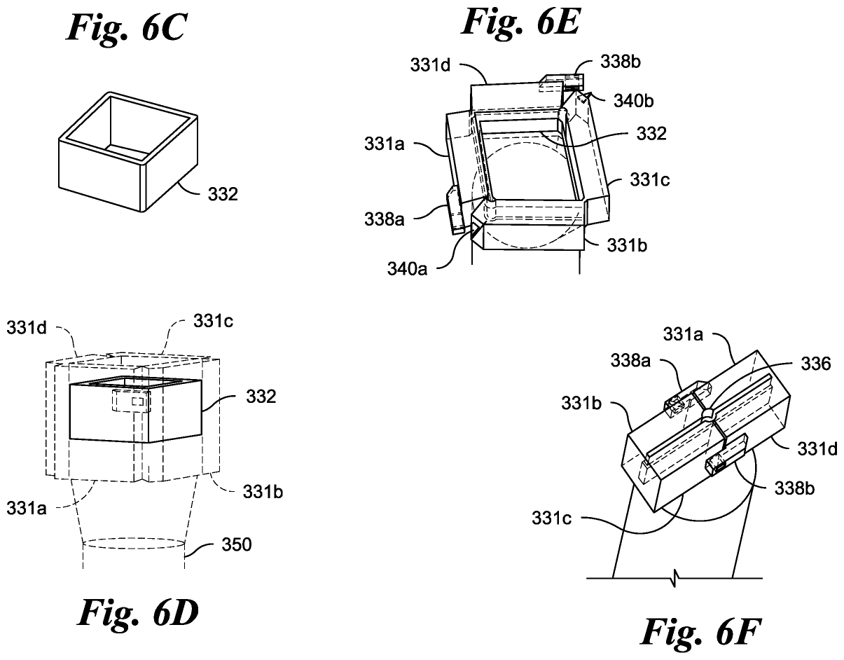

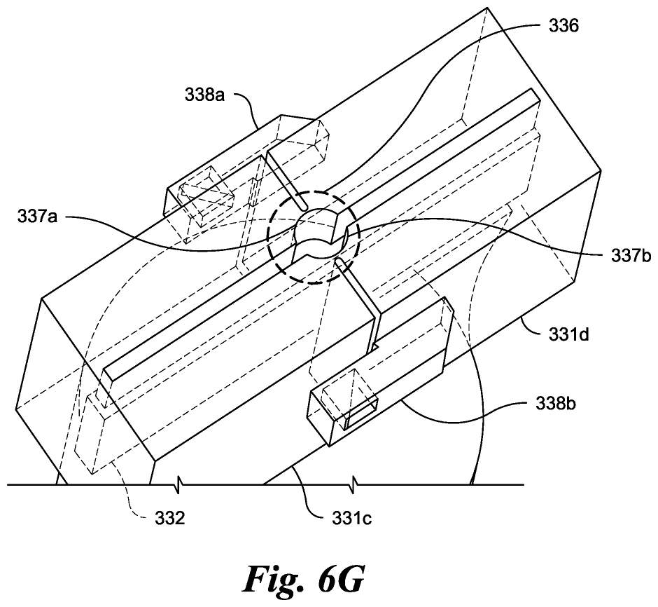

According to another embodiment, the stopper of the container assembly may be replaced, with a collapsible means that is configured to switch from an open condition to a sealed condition. For example, referring to FIGS. 6A to 6G, in one embodiment, the container assembly 310 comprises a collar 330 having a plurality of connected segments, preferably in the form of panels 331a, 331b, 331c, 331d. In an embodiment in which the segments comprise four panels, such as the embodiment illustrated in FIGS. 6A to 6G, the opening within the collar 330 may, take the form of a rectangle, square, or parallelogram in the initial open configuration. The segments of the collar are preferably hingedly connected, as by a hinge or a living hinge, for example, and made of similar materials as the stopper collars of the previously described embodiments. The container assembly 310 may Maher comprise a flexible film 350, made of similar materials and preferably transparent as previously described, that is attached to one of the open ends of the collar 330 and dosed at a distal end 351 of the film 350 to essentially form a tillable bag or pouch. A rigid frame as previously disclosed may be incorporated within the container assembly 310; however, the rigid frame is optional.

While the collar 330 is in the open condition, the container assembly 310 may be filled with a therapeutic liquid and subsequently sealed by collapsing the collar 330 along pre-defined bending/deflection zones that effectively hinge the assembly collar 330 to permit movement into a relatively flat closed configuration, as illustrated in FIGS. 6B, 6F and 6G. When the collar 330 has been moved into the closed condition, a closure latch 338a located on panel 331a may capture catch 340a located on panel 331b to secure the collar in the dosed condition. Optionally, a second closure latch 338b located on panel 331d and catch 340h located on panel 331c may be located on the opposing side of the collar 330 in the closed condition.

The collar 330 may further comprise a seal portion 332 located on at least a portion of the inner surface of the panels 331a-331d. When the collar 330 is in the dosed condition, the seal portion 332 may similarly be compressed into a at configuration. The seal portion 332 is preferably made of material that may be sealed under sufficient temperature and/or pressure to seal the container assembly 310 and thereby ensure container closure integrity during the entire product life of the therapeutic liquid within the container. For example, the material of the seal portion 332 may be an elastomer, pressure sensitive adhesive, or other material that is pliable and capable of forming a seal by being compressed against itself with sufficient pressure by the panels 331a-331d. Additionally, a port 336 (best viewed in FIG. 6G) may preferably be provided through a central portion of the collar 330 (when collapsed) and may preferably be formed by curved portions 337a, 337b of the respective panels 331a-331d which are aligned when the collar 330 is collapsed. The port 336 may be provided as a guide for a needle that may be introduced through the sealing elastomer of the seal portion 332 for extraction of the therapeutic liquid from within the container.

The embodiment depicted in FIGS. 6A-6G may be include one or more of the previously disclosed features, such as the frames and/or needle hubs depicted in FIGS. 1-4. In certain embodiments, a needle hub is not used; and once the container is filled and container closure is established, the fluid may be extracted by introducing a needle through a port (such as the port 336 in FIGS. 6f and 6g) and through the elastomer of the seal portion. Alternately, embodiments with an integrated needle may utilize the needle as an outlet to expel the contents of the container assembly. Pressure applied to the flexible film 350 may generate an internal pressure serving to expel the therapeutic liquid from the container assembly 310. Embodiments including a structural frame may provide containers that may be more easily handled within filling equipment or loaded into nesting tubs, for example, as well as loading into injection devices comprising mechanical means for applying pressure to the flexible film.

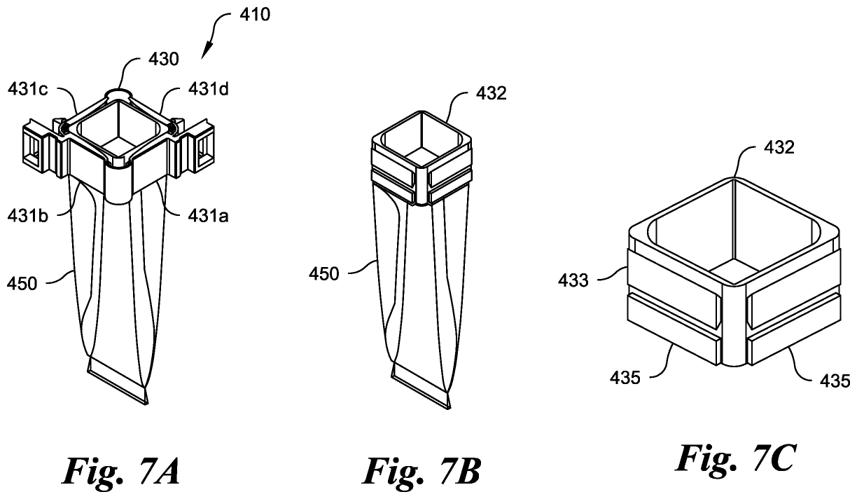

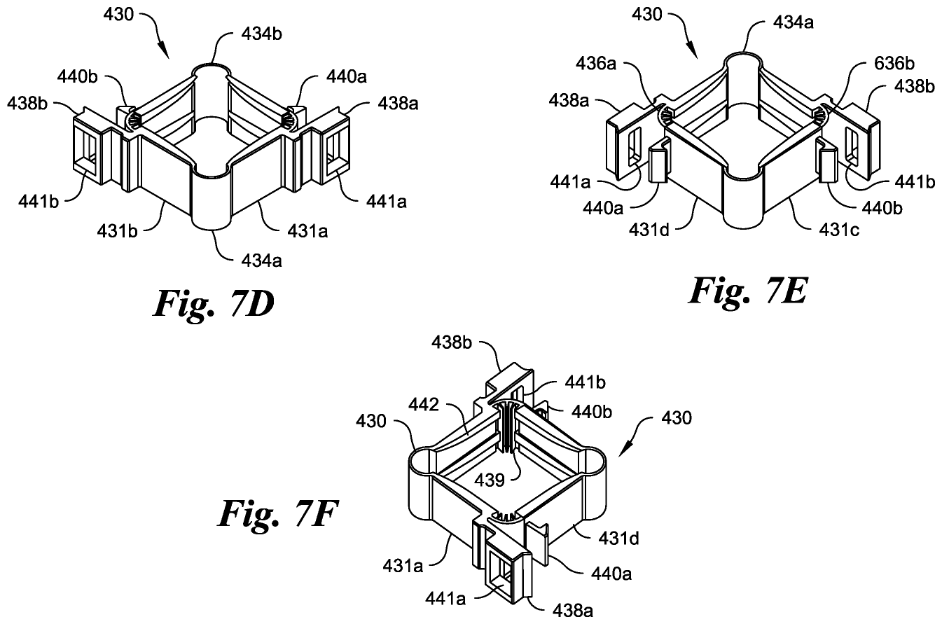

FIGS. 7A to 7F are views of another embodiment of a container assembly 410 having a flexible film 450, preferably transparent film, attached to a collapsible collar 430 shown in an open configuration. Both the film 450 and collar 430 may be made of similar materials as previously described. The collar 430 comprises a plurality of segments, preferably in the form four rectangular panels, 431a, 431b, 431c, 431d, with the panels being connected, preferably by hinges 434a, 434b, 436a, 436b (which may be living; hinges formed from a portion of the panel material) at adjacent panel edges such that the collar 430 may be collapsed into a closed configuration. Similar to the embodiment of FIGS. 6A to 6G, the collar 430 includes latches 438a, 438b and catches 440a, 440b on opposing sides of the collar 430. When collapsed to a closed state, hinges 436a, 436b, which are located between the latches 438a, 438b and catches 440a, 440b are urged towards each other until the catches 440a, 440b pass through an aperture 441a, 441b of their respective latches 438a, 438b to maintain the collar 430 in a flat closed configuration.

The collar 430 further comprises a seal portion 432 that is attached to the inner surfaces of the panels 431a, 431b, 431c, 431d. Similar to the previously described seal portion, the seal portion 432 is preferably made of a material that is capable of being sealed under sufficient pressure and/or temperature. To ensure that sufficient compression is applied to the portions of the seal portion 432 adjacent to the hinges 436a, 436b between the latches 438a, 438b and catches 440a, 440b, the hinges 436a, 436b may be provided with one or more longitudinal fins 439 that will contact and compress the central portions of the seal portion 432 in the flat closed configuration.

As best viewed in FIG. 7B, the flexible film 450 may be attached to the collar 430 between the panels 431a, 431b, 431c, 431d and the seal portion 432. The film 450 is preferably fused to the lower portion of the outer surface of the seal portion 432. In order to affix the seal portion 432 to the inner surfaces of the panels 431a, 431b, 431c, 431d, the seal portion may be provided with one or more rails 433 that may be inserted or molded into corresponding tracks 442 on the inner surfaces of the panels 431a, 431b, 431c, 431d. The edges of the rails 433 may be flared in order to provide an interference fit, for example, between the rails 433 and the tracks 442.

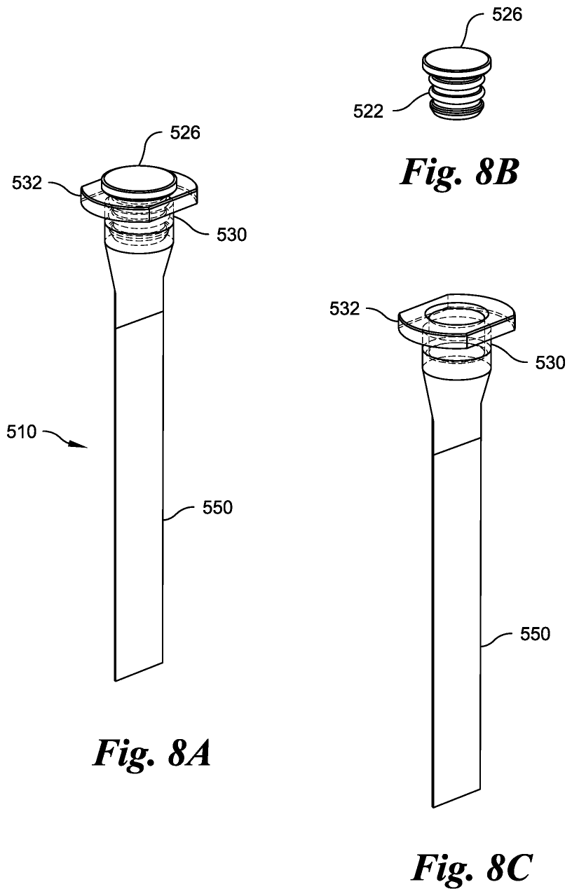

FIGS. 8A to 8C are views of a container assembly 510 having a collar 530 with a rectangular radially outwardly extending flange 532 and a stopper 526 having radial ridges 522 for forming a seal with the inner surface of the collar 530. The container assembly 510 further comprises a flexible film 550. As illustrated, film 550 is collapsed; therefore, the container assembly 510 is shown in the empty condition, after use, A rigid frame may or may not be included in the container assembly 510. The collar 530 is provided with a relatively large flange 532, so that the container assembly 510 may be loaded into automated filling equipment and/or nest tubs, for example.

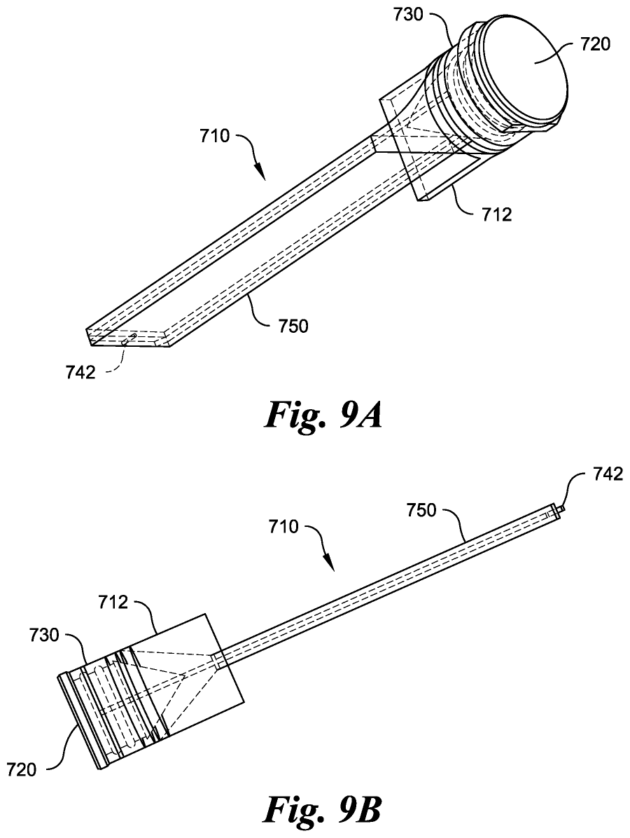

Referring to FIGS. 9A and 9B, another embodiment of the container assembly 710 includes a collar 730 compatible with a tapered stopper 720. The container assembly 710 is shown in a collapsed state, in which the film 750 and the container 710 is generally rectangular. The container 710 does not have a needle hub or needle attached, but may include an optional port 742 located within a distal end of the rectangular frame within the film 750. Therefore, the liquid contents of the container assembly 710 may be accessed either through the stopper 720 or the port 742. The container assembly 710 has a collar for receiving the stopper 720, and the frame within the film 750 is attached to a distal end of the collar 730. The collar 730 may also include a flange 712. Unlike the previously described flanges that extend radially from a proximal end of the collar, the collar 730 may include an axially oriented flange 712 arranged generally perpendicular to a principal side of film 750 wherein the rectangular shape is formed when the container is collapsed after use. The axially oriented flange 712 provides two corners having an axial edge and a radial edge that may aid in aligning the container 710 within an injection device or other fluid-dispensing device.

Referring to FIGS. 10A to 10D, another embodiment of a container assembly 810 includes a stopper collar 830, a stopper 820, a film 850, and a Y-shaped internal frame 840 with a needle hub 860 and a needle 870 inserted therein. The Y-shaped internal frame 840 has a proximal end 840a connected to the stopper collar 830 and a distal end 840b connected to the needle nub 860. The frame 840 has two outer legs 840c, 840d which taper from two outer-leg connection points on the bottom surface at the distal end of the collar 830 inwardly to join an intermediate portion 840e of the Y-shaped frame. In the embodiment depicted, the intermediate portion 840e has a smaller width than the diameter of the stopper collar 830. The internal frame 840 also has a center leg 840f which connects to a transverse support member 840g of the stopper collar. An aperture 844 located towards the distal end 840b of the frame 840 may be in fluid communication with a via 848 and the needle 870 to provide a pathway for fluid to exit the container through the central lumen of the needle 870. Note that in any of the embodiments, the support frames preferably may be generally planar so that when the container is empty, the film may collapse as much as possible.

It will be appreciated by those skilled in the art that changes could be made to the embodiments described above without departing from the broad inventive concept thereof. It is understood, therefore, that the scope of this disclosure is not limited to the particular embodiments disclosed, but it is intended to cover modifications within the spirit and scope thereof

I claim:

1. A container assembly for a therapeutic liquid comprising:
a frame having a proximal end and a distal end and comprising a plurality of axially extending legs;
a collar attached to the plurality of axially extending legs at the proximal end of the frame; and
a collapsible film covering the frame and attached to the distal end of the frame and at least a portion of the collar.

2. The container assembly of claim 1 further comprising an elastomeric closure, wherein the collar is configured to receive the elastomeric closure.

3. The container assembly of claim 2, wherein the elastomeric closure comprises a material selected from the group consisting of butyl rubber, a thermoplastic elastomer, and thermoplastic urethane.

4. The container assembly of claim 2, wherein the elastomeric closure comprises at least one radial ridge around an outer circumferential surface of the elastomeric closure.

5. The container assembly of claim 2, wherein the collar comprises at least one captivating ridge around an inner circumferential surface of the collar configured to captivate the elastomeric closure within the collar.

6. The container assembly of claim 2, wherein the elastomeric closure comprises a tapered end.

7. The container assembly of claim 1, wherein the collar includes a flange.

8. The container assembly of claim 1, wherein the film is transparent.

9. The container assembly of claim 1, wherein the film is attached to the frame and the collar.

10. The container assembly of claim 1 further comprising a hub integral with and extending from the distal end of the frame.

11. The container assembly of claim 7 further comprising a needle axially extending through the hub.

12. The container assembly of claim 1, wherein the frame is rectangular.

13. The container assembly of claim 1, wherein at least one of the collar and the frame comprise a material selected from the group consisting of a cyclic olefin polymer, cyclic olefin copolymer, polypropylene, and glass.

14. The container assembly of claim 1, wherein the collapsible film includes a cylindrical portion between the proximal and distal ends of the frame.

15. The container assembly of claim 1, wherein a portion of the collapsible film proximate to the distal end of the frame is tapered.

16. A plurality of container assemblies according to claim 1 in combination with a nest tub, wherein the plurality of container assemblies is arranged within the nest tub.

17. A container assembly for a therapeutic liquid comprising:
a collar comprising a plurality of hingedly connected segments configured to switch from an open configuration to a closed configuration; and
a collapsible film configured as a pouch having an open end attached to one end of the collar.

18. The container assembly of claim 17, wherein a first segment of the plurality of segments is configured to form a seal with a second segment of the plurality of segments when the collar is in the closed configuration.

19. The container assembly of claim 17, wherein the collar further comprises a sealing material attached to at least one of the inner surfaces of the segments.

20. The container assembly of claim 17, wherein the collar further comprises a needle port in the closed configuration.
